(19) United States
(12) Patent Application Publication
(10) Pub. No.: US 2026/0043767 A1
(43) Pub. Date: Feb. 12, 2026

(54) MAGNETIC BEAD ELISA DETECTOR DEVICE AND METHODS OF USE THEREOF

(71) Applicant: CARDIAI TECHNOLOGIES LTD., Calgary, Alberta (CA)

(72) Inventors: Margaret RENAUD-YOUNG, Calgary, Alberta (CA); Azam SAYADI, Calgary, Alberta (CA); Anmol KAPOOR, Calgary, Alberta (CA); Raja Balraj SINGH, Calgary, Alberta (CA); Viola Ingrid BIRSS, Calgary, Alberta (CA)

(21) Appl. No.: 18/998,994

(22) PCT Filed: Jul. 28, 2023
(86) PCT No.: PCT/CA2023/051020

§ 371 Date: Jan. 27, 2025

Related U.S. Application Data
(60) Provisional application No. 63/393,677, filed on Jul. 29, 2022.

(51) Int. Cl. G01N 27/327; B03C 1/01; B03C 1/30; G01N 27/30; G01N 33/543

(52) U.S. Cl. CPC G01N 27/3276; B03C 1/01; B03C 1/30; G01N 27/308; G01N 27/3272; G01N 27/3277; G01N 33/5438; B03C 2201/26

(57) ABSTRACT

Provided are systems, cartridges, devices and methods for detecting a target analyte in a test sample. The system employs a multitude of magnetic sources for moving magnetic beads in a sequential movement through various portions of e.g. a cartridge or strip, such as from one or more hybridization portions, through one or more washing portions, and to a redox portion. The system employs an electrode set and a potentiostat for performing amperometry to obtain an electrochemical measurement based on conversion of a redox active molecule. Also provided are methods for detecting a target analyte using the systems, and cartridges and strips for use in the system or a device of the system.

### CROSS-REFERENCE TO RELATED APPLICATION

This application claims priority to and benefit from U.S. Patent Application Ser. No. 63/393,677 filed on Jul. 29, 2022, which is hereby incorporated by reference in its entirety.

### FIELD OF THE DISCLOSURE

The present disclosure relates generally to systems, devices, cartridges, strips, and methods for detecting a target analyte in a test sample, and in particular, to a system or device employing a multitude of magnetic sources for moving magnetic beads in a sequential movement and a potentiostat for performing amperometry to obtain electrochemical measurements.

### BACKGROUND

Enzyme linked immunosorbent assay (ELISA) is used in many different areas of analytical chemistry and medicine and is capable of testing and providing semi-quantitative measurements for a wide variety of target analytes or compounds of interest in a given test sample, such as an antigen, a toxin, a hormone, a protein, or a specific biomarker in blood or plasma.

However, currently available ELISA testing and devices are not ideal for detecting the presence or precise quantities of certain analytes, for example due to their low concentrations, and/or the detection efficiency is inadequate. Moreover, ELISA tests are often too complex for general point of care (POC) testing and home testing. There is also lack of sensitivity for detecting the presence of certain analytes in low concentrations and lack of quantitative measurement capability in currently available lateral flow tests. Devices and methods having improved performance, sensitivity and other characteristics are thus needed.

It is an object of the present disclosure to provide systems, components thereof, and methods for detecting target analytes in a test sample.

### SUMMARY

The present disclosure relates to systems, devices, cartridges, strips and methods for detecting an analyte in a test sample.

According to one aspect of this disclosure, there is provided a system for detecting a target analyte in a test sample, the system comprising: a test sample receiving portion; one or more hybridization portions interconnected to the test sample receiving portion, wherein the one or more hybridization portions comprise or are capable of being loaded with: a first antibody coupled to a magnetic bead, the first antibody capable of specifically binding to the target analyte, and a second antibody coupled to an enzyme, the second antibody capable of specifically binding to the target analyte, wherein the enzyme is capable of converting a redox active molecule; a redox portion interconnected to the one or more hybridization chambers, wherein the redox portion comprises or is capable of being loaded with a developing solution having the redox active molecule; an electrode set; a potentiostat for performing amperometry to obtain an electrochemical measurement based on conversion of the redox active molecule; and a multitude of magnetic sources positioned for moving the magnetic bead in a sequential movement from the hybridization portion to the redox portion and optionally beyond, wherein different magnetic sources of the multitude of magnetic sources are capable of being turned on and off in a desired sequence to achieve the sequential movement of the magnetic bead.

In an embodiment, the system comprises a cartridge or a strip that is insertable and removable from a device, the cartridge or strip comprising the test sample receiving portion, the one or more hybridization portions, the redox portion, and the electrode set; and the device comprising the potentiostat and the multitude of magnetic sources.

In an embodiment, the device further comprises a slot or an opening for receiving the cartridge or strip. In an embodiment, the device comprises one or more piercing components.

In an embodiment, the cartridge comprises: a port or receptacle as the test sample receiving portion; one or more hybridization chambers as the one or more hybridization portions, interconnected to the port or receptacle by a first passage; a redox chamber as the redox portion interconnected to the one or more hybridization chambers by a second passage; and the electrode set.

In an embodiment, the cartridge further comprises one or more valves capable of being opened and closed, the one or more valves located within the first passage, the second passage, or both.

In an embodiment, the first passage, the second passage, or both, is a tubing or a microchannel.

In an embodiment, the strip comprises an absorbent pad as the test sample receiving portion.

In an embodiment, the strip is a disposable strip.

In an embodiment, the first antibody and/or the second antibody are in a hybridization solution, and the hybridization solution is contained in a hybridization pouch or blister capable of being pierced to release the hybridization solution into or onto the one or more hybridization portions.

In an embodiment, the one or more hybridization portions comprise: a first hybridization portion interconnected to the test sample receiving portion and comprising or capable of being loaded with the first antibody; and a second hybridization portion interconnected downstream to the first hybridization portion and comprising or capable of being loaded with the second antibody.

In an embodiment, the system further comprises a washing portion positioned between the one or more hybridization portions and the redox portion, the washing portion comprising or capable of being loaded with a washing solution.

In an embodiment, the system further comprises a washing portion positioned along the second passage between the one or more hybridization portions and the redox portion, the washing portion comprising or capable of being loaded with a washing solution.

In an embodiment, the washing portion is a washing chamber.

In an embodiment, the washing solution is contained in a washing pouch or blister capable of being pierced to release the washing solution into or onto the washing portion.

In an embodiment, the washing solution is an oil or an organogel.

In an embodiment, the developing solution is contained in a developing pouch or blister capable of being pierced to release the developing solution into or onto the redox portion.

In an embodiment, the system further comprises one or more piercing components for piercing the pouch or the blister.

In an embodiment, the test sample receiving portion comprises a size selective mesh for capturing cells or molecules larger than a defined size.

In an embodiment, the defined size of the size selective mesh is between about 0.5 and about 10 μm, and more particularly about 4.5 μm.

In an embodiment, the test sample receiving portion or the one or more hybridization portions comprise an absorbent pad for absorbing a fluid and transferring it into the one or more hybridization portions.

In an embodiment, the electrode set comprises a three-electrode cell.

In an embodiment, the three-electrode cell comprises a reference electrode, a counter electrode and a working electrode.

In an embodiment, the working electrode and the counter electrode are comprised of a carbon ink, the reference electrode is comprised of Ag, AgCl or the carbon ink, and optionally the working electrode is further coated with an acid. In a particular embodiment, the working electrode and the counter electrode are both comprised of the carbon ink, and the reference electrode is comprised of an Ag, AgCl ink.

In an embodiment, the magnetic sources are electromagnets.

In an embodiment, the magnetic sources are positioned in a zig zag pattern along a path of the sequential movement.

In an embodiment, the system further comprising a Faraday cage separating the electrode set and/or the potentiostat from the multitude of magnetic sources.

In an embodiment, the potentiostat comprises a potentiostat circuitry that electrically engages the electrode set for performing amperometry to obtain the electrochemical measurement.

According another aspect of this disclosure, there is provided a method for detecting a target analyte in a test sample, the method comprising: providing the system as described herein; contacting a test sample with the test sample receiving portion for receiving the test sample; allowing the test sample to migrate from the test sample receiving portion to the one or more hybridization portions; contacting the test sample, within the one or more hybridization portions, with the first antibody and the second antibody; sequentially activating the different magnetic sources of the multitude of magnetic sources to move the magnetic bead coupled to the first antibody from the one or more hybridization portions to the redox portion; contacting the magnetic beads in the redox portion with a developing solution having the redox active molecule; performing amperometry using the potentiostat to obtain an electrochemical measurement from the electrode set based on conversion of the redox active molecule by a redox reaction; and determining or evaluating the presence, absence and/or quantity of the target analyte based on the electrochemical measurement.

In an embodiment, the step of allowing the test sample to migrate from the test sample receiving portion to the one or more hybridization portions comprises passing the test sample through a size selective mesh.

In an embodiment, the method comprises a step of piercing one or more hybridization pouches or blisters to release the first antibody and/or second antibody into or onto the one or more hybridization portions.

In an embodiment, the method further comprises one or more steps of washing the magnetic beads with a washing solution, the one or more washing steps being performed within a washing portion positioned between the one or more hybridization portions and the redox portion.

In an embodiment, the washing solution comprises an oil or an organogel.

In an embodiment, the one or more steps of washing comprise piercing a washing pouch or blister to release the washing solution into or onto the washing portion.

In an embodiment, the method further comprises a step of piercing a developing pouch or blister to release the developing solution into the redox portion.

In an embodiment, the step of sequentially activating the different magnetic sources of the multitude of magnetic sources comprises sequentially activating the different magnetic sources in a zig zag pattern along a path of the sequential movement.

In an embodiment, the method further comprises a step of opening and/or closing one or more valves to aid in sequential movement of the magnetic bead.

In an embodiment, the step of performing amperometry comprises a step of introducing a stopper solution to the redox portion.

In an embodiment, the stopper solution comprises an acid solution.

In an embodiment, the acid solution comprises sulfuric acid.

In an embodiment, the step of performing amperometry comprises flowing the contents of the redox portion to an electrode chamber, passage or portion that comprises, or is operationally associated with, a working electrode of the electrode set.

In an embodiment, the working electrode comprises an acid solution for stopping or quenching the redox reaction. In an embodiment, the working electrode contains or is coated with an acid solution for stopping or quenching the redox reaction.

In an embodiment, the step of performing amperometry is performed for 30 seconds.

According to another aspect of this disclosure, there is provided a disposable cartridge comprising: a port or receptacle for receiving a test sample; one or more hybridization chambers interconnected to the port or receptacle by a first passage, wherein the one or more hybridization chambers comprise within or are capable of being loaded with: a first antibody coupled to a magnetic bead, the first antibody capable of specifically binding to the target analyte, and a second antibody coupled to an enzyme, the second antibody capable of specifically binding to the target analyte and the enzyme is capable of converting a redox active molecule, wherein the first antibody and the second antibody are from an antibody source located on the cartridge; a redox chamber interconnected to the one or more hybridization chambers by a second passage, the redox chamber comprising within or capable of being loaded with a developing solution having the redox active molecule; and an electrode set; wherein the cartridge is configured for sequential movement of the magnetic bead from the hybridization chamber to the redox chamber and optionally beyond, by a multitude of magnetic sources present in a device capable of housing the cartridge.

According to another aspect of this disclosure, there is provided a disposable strip comprising: a test sample receiving portion; one or more hybridization portions downstream of the test sample receiving portion, wherein the one or more hybridization portions comprise or are capable of being loaded with: a first antibody coupled to a magnetic bead, the first antibody capable of specifically binding to the target analyte, and a second antibody coupled to an enzyme, the second antibody capable of specifically binding to the target analyte and the enzyme is capable of converting a redox active molecule; wherein the first antibody and the second antibody are from an antibody source located on the strip; a redox portion downstream of the hybridization portion, the redox portion comprising or capable of being loaded with a developing solution having the redox active molecule; and an electrode set; wherein the strip is configured for sequential movement of the magnetic bead from the hybridization chamber to the redox chamber and optionally beyond, by a multitude of magnetic sources present in a device capable of housing the strip.

According to another aspect of this disclosure, there is provided a kit for detecting a target analyte in a test sample, the kit comprising: the device as disclosed herein and instructions for using the device with a cartridge or strip as disclosed herein. In an embodiment, the kit further comprises one or more cartridges or strips as disclosed herein. In an embodiment, the kit further comprises one or more buffers or solutions, including for example solutions for solubilizing or suspending the test sample, solutions comprising the first and/or second antibody, wash buffers or solutions, redox buffers or solutions, solutions containing an acid, or any combination thereof.

### BRIEF DESCRIPTION OF THE DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 is a perspective view of a cartridge with a linear design, for detecting a target analyte in a test sample, according to one embodiment of this disclosure;

FIG. 2 is a cross-sectional view of a cartridge with pouches or blisters capable of being pierced to release the content inside the pouches or blisters;

FIG. 3 is a perspective view of a cartridge with a circular design, for detecting a target analyte in a test sample, according to another embodiment of this disclosure;

FIG. 4 is a cross-sectional view of a strip, for detecting a target analyte in a test sample, according to another embodiment of this disclosure;

FIG. 5 is a perspective view of a system showing the cartridge depicted in FIG. 1 and a device with a slot for receiving the cartridge, for detecting a target analyte in a test sample, according to one embodiment of this disclosure;

FIG. 6 is a cross-sectional view of the device depicted in FIG. 4; and

FIG. 7 is a flowchart showing the steps of a process for detecting a target analyte in a test sample, according to one embodiment of this disclosure.

### DETAILED DESCRIPTION

The present disclosure relates to systems, devices, cartridges, strips and methods for detecting a target analyte in a test sample. The systems, devices, cartridges, strips and methods comprise improved design and features.

Advantageously, the system and device disclosed herein comprise a multitude of magnetic sources located throughout (e.g. on a device of the system) for moving the magnetic beads in a sequential movement, and in select embodiments creating a swishing and/or mixing movement. Different magnetic sources of the multitude of magnetic sources are capable of being turned on and off in a desired sequence to achieve the sequential movement. During the sequential movement, and in certain embodiments in relation to the swishing and/or mixing movement of the magnetic beads and increased travel distance, improved hybridization may be achieved for example by a rapid and efficient mixing, and improved washing may also be achieved with the fluid containing uninterested materials from the test sample being removed as only the magnetic bead coupled to a first antibody which binds to the target analyte move through the system (e.g. on the cartridge or strip). This also may create an improved redox reaction, again, with rapid and efficient mixing of the magnetic beads coupled to the first antibody and the enzyme coupled to the second antibody, with the redox active molecule in the redox solution. This directed path of sequential movement can be designed and achieved by turning on and off the magnetic sources, such as for example in a zig zag pattern. In select embodiments, this zig zag pattern provides an improved and highly efficient system for detection of a target analyte.

### Systems, Devices, Cartridges, Strips, and Kits

In an embodiment, the present disclosure relates to a system for detecting a target analyte in a test sample, the system comprising: a test sample receiving portion; one or more hybridization portions interconnected to the test sample receiving portion, wherein the one or more hybridization portions comprise or are capable of being loaded with: a first antibody coupled to a magnetic bead, the first antibody capable of specifically binding to the target analyte, and a second antibody coupled to an enzyme, the second antibody capable of specifically binding to the target analyte, wherein the enzyme is capable of converting a redox active molecule; a redox portion interconnected to the one or more hybridization chambers, wherein the redox portion comprises or is capable of being loaded with a developing solution having the redox active molecule; an electrode set; a potentiostat for performing amperometry to obtain an electrochemical measurement based on conversion of the redox active molecule; and a multitude of magnetic sources positioned for moving the magnetic bead in a sequential movement from the hybridization portion to the redox portion and optionally beyond, wherein different magnetic sources of the multitude of magnetic sources are capable of being turned on and off in a desired sequence to achieve the sequential movement of the magnetic bead.

In an embodiment, the system is a device that comprises all of the components described above in a fixed unit, namely it comprises each of the test sample receiving portion, the one or more hybridization portions, the redox portion, the electrode set; the potentiostat and the multitude of magnetic sources in a fixed unit. By “fixed unit”, it is meant that system operates as a single unit without there being a removable cartridge or strip. In such embodiment, the entirety of the system may be of a single use design and may be disposable.

In an embodiment, the system comprises a cartridge or a strip that is insertable and removable from a device, the cartridge or strip comprising the test sample receiving portion, the one or more hybridization portions, the redox portion, and the electrode set; and the device comprising the potentiostat and the multitude of magnetic sources.

In an embodiment, the device comprises a slot or an opening for receiving the cartridge or strip. The slot or opening may be of any design or configuration that is suitable for receiving the cartridge or strip. In an embodiment, the cartridge or strip is inserted in a manner that it is positioned internal within the device. In an embodiment, the cartridge or strip is inserted in a manner that it is positioned on an exterior surface of the device. In an embodiment, the cartridge or strip is inserted in a manner that it is positioned partially internal within the device and partially on an exterior surface of the device. In an embodiment, the device is capable of acknowledging that a strip or a cartridge is inserted correctly into the slot or the opening of the device, and is capable of providing an alert and/or an error message to a user if the strip or cartridge is inserted incorrectly. In another embodiment, the device further comprises a timer, for example configured to start when the test sample is added to the sample receiving portion and thereafter timing the progress continuously as the test sample moves through the device and various portions and/or chambers.

As used herein, the term “target analyte” refers to any compound, molecule, or substance which is desired to be detected in a test sample. Examples of target analytes include, without limitation, an antigen, a protein, a peptide, a toxin, a drug, a metabolite, an antibody, a hormone, a vitamin, a cytokine, a microorganism (e.g. bacteria, virus, or protozoa), or the like. The test sample may, for example, be any bodily fluid, such as blood, serum, plasma, or urine.

As used herein, by “test sample receiving portion”, it is meant to be the portion where the test sample is deposited or comes into contact with the system (e.g. on a cartridge or a strip). The test sample receiving portion may be of any size or shape. The test sample receiving portion may be made of any suitable material to receive the test sample and permit fluid flow, e.g. by capillary action. In an embodiment, wherein the system contains a cartridge, the test sample receiving portion may be a port or receptacle. In another embodiment, wherein the system contains a strip, the test sample receiving portion may be an absorbent pad.

In embodiments in which the system comprises an insertable and/or removable cartridge or strip, the test sample may be loaded onto the cartridge or strip before or after insertion of the cartridge or strip into a device of the system. In an embodiment, the test sample is loaded onto the cartridge or strip before insertion of the cartridge or strip into a device of the system.

In an embodiment, the test sample receiving portion comprises a size selective mesh for prohibiting materials including cells or molecules larger than a defined size to pass through. In an embodiment, the size selective mesh has a defined size of between about 0.5 μm to about 10.0 μm. In an embodiment, the size selective mesh has a defined size of about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, about 5.5 μm, about 6.0 μm, about 6.5 μm, about 7.0 μm, about 7.5 μm, about 8.0 μm, about 8.5 μm, about 9.0 μm, about 9.5 μm, or about 10.0 μm. In an embodiment, the size selective mesh has a defined size of about 4.5 μm.

Different mesh materials may be used to trap or prohibit undesired materials or test sample components from passing through. For example, the size selective mesh may have a defined size that absorbs and does not allow certain or any large complexes or biomolecules (e.g. of a megadalton size) to pass through, but does not capture a particular target analyte. Those skilled in the art will appreciate that various alternative materials or mesh sizes are readily available.

The system of the present disclosure comprises one or more hybridization portions. As used herein, the “hybridization portion” is intended to refer to a portion in which the test sample is able to come into contact with one or more antibodies as described herein. As described herein, in an embodiment the hybridization portion may be one or more hybridization chambers. In another embodiment, the hybridization portion may be a region or regions on a strip. In an embodiment, there is a single hybridization portion where the test sample comes into contact with both a first antibody and a second antibody as described herein. In an embodiment, there are multiple hybridization portions where the test sample comes into contact with the first antibody and a second antibody in different hybridization portions.

In an embodiment, the one or more hybridization portions comprise a first hybridization portion interconnected to the test sample receiving portion and comprising within or capable of being loaded with the first antibody; and a second hybridization portion interconnected downstream to the first hybridization chamber and comprising within or capable of being loaded with the second antibody.

As described elsewhere herein, the first antibody is one that is coupled to a magnetic bead and is capable of specifically binding to the target analyte and the second antibody is one that is coupled to an enzyme and is also capable of specifically binding to the target analyte. The enzyme is capable of converting a redox active molecule. In particular, the enzyme is capable of converting a redox molecule to a form that changes its “energy properties” and that redox molecule later is detected with the potentiostat.

In an embodiment wherein the system includes a cartridge, the one or more hybridization portions may be one or more chambers and the test sample receiving portion may be a port or receptacle. The one or more hybridization chambers may be interconnected to the port or receptacle by any suitable means. In an embodiment, the one or more hybridization chambers may be interconnected to the port or receptacle by a first passage (e.g. tubing or conduit) to allow the test sample to move from the port or receptacle to the one ore more hybridization chambers.

As used herein, the term “passage” is intended to refer to any conduit, channel or other structure that allows material to pass through and to link the various portions and chambers of the device together. In an embodiment, the passage is a tubing or a microchannel.

In an embodiment wherein the system includes a strip, the one or more hybridization portions may be regions on the strip downstream in a flow path from the test sample receiving portion. In such embodiments, by “interconnected” it is meant that the test sample is able to flow from one portion or region of the strip to another. The first antibody and second antibody described herein may be within the same region of the strip such that the test sample contacts both simultaneously, or one may be located downstream in the flow path from the other such that the test sample contacts each sequentially.

In an embodiment, the first antibody and the second antibody may already be in the one or more hybridization portions at the time the test sample reaches these portions. In an embodiment, the first antibody and the second antibody may be in a hybridization solution or hybridization buffer. In an embodiment, the first antibody and the second antibody may be in a lyophilized state. In embodiments in which the first antibody and the second antibody are in a lyophilized state, a hybridization solution or hybridization buffer may be added to the one or more hybridization portions before, during or after the test sample being introduced to the one or more hybridization portions. In an embodiment, the hybridization solution or hybridization buffer is contained in a pouch or blister that can be opened or pierced to release the hybridization solution or hybridization buffer into or onto the one or more hybridization portions.

In other embodiments, first antibody and the second antibody may be contained within a hybridization pouch or blister that is opened or pierced to release the first antibody and second antibody into (e.g. for chambers) or onto (e.g. for strips) the one or more hybridization portions. In embodiments where the one or more hybridization portions are chambers, the pouch or blister may be internal or external to the chamber, so long as the first antibody and second antibody are capable of being released into the chamber. Within the pouch or blister, the first antibody and second antibody may be already in a hybridization solution or buffer, or upon opening or piercing a hybridization solution or buffer is released to solubilize or suspend the first antibody and second antibody.

As used herein, the term “pouch” or “blister” is intended to refer to any enclosed structure capable of being opened or pierced to release its contents. Those skilled in the art will appreciate that various materials for the pouch or blister are readily available. In an embodiment, the system (e.g. the device) further comprises piercing components for opening or piercing the pouches or the blisters as described herein to release the contents.

In an embodiment, the test sample receiving portion or the one or more hybridization portions further comprise an absorbent pad for absorbing a fluid and transferring it into the one or more hybridization portions. The absorbent pad may comprise the size selective mesh as described elsewhere herein. The absorbent pad may also absorb or trap components of the test sample that are undesirable for movement through the system. If effect, this may provide an efficient wash effect, even without a washing portion component (as described elsewhere herein).

The system of the present disclosure comprises a redox portion. As used herein, the “redox portion” is intended to refer to a portion of the system in which the magnetic bead complex is brought into contact with a developing solution having redox active molecules. By “magnetic bead complex”, it is meant to refer to the complex that is formed in the one or more hybridization portions. In situations where the target analyte is absent from the test sample, the magnetic bead complex should include only the first antibody with the coupled magnetic bead. In situations where the target analyte is present in the test sample, the magnetic bead complex should include the first antibody with the coupled magnetic bead, the target analyte, and the second antibody with the coupled enzyme. The redox portion comprises or is capable of being loaded with a developing solution having the redox active molecule.

In an embodiment wherein the system includes a cartridge, the redox portion may be a redox chamber. The redox chamber may be interconnected to the one or more hybridization chambers by any suitable means. In an embodiment, the redox chamber may be interconnected to the one or more hybridization chambers by a second passage (e.g. tubing or conduit) to allow any magnetic bead complex to move from the one or more hybridization chambers to the redox chamber.

In an embodiment wherein the system includes a strip, the redox portion may be a region on the strip downstream in the flow path from the one or more hybridization portions. In such embodiments, by “interconnected” it is meant that the magnetic bead complex is able to flow from one portion or region of the strip to another.

In an embodiment, the redox active molecule may already be in the redox portion at the time the magnetic bead complex reaches this portion. In an embodiment, the redox active molecule may be in a redox developing solution or developing buffer. In an embodiment, the redox active molecule may be in a lyophilized state. In embodiments in which the redox active molecule is in a lyophilized state, a redox developing solution or developing buffer may be added to the redox portion before, during or after the magnetic bead complex is introduced to the redox portion. In an embodiment, the redox developing solution or developing buffer is contained in a pouch or blister that can be opened or pierced to release the redox developing solution or developing buffer into or onto the redox portion.

In other embodiments, redox active molecule may be contained within a redox pouch or blister that is opened or pierced to release the redox active molecule into (e.g. for chambers) or onto (e.g. for strips) the redox portion. In embodiments where the redox portion is a chamber, the pouch or blister may be internal or external to the chamber, so long as the redox active molecule is capable of being released into the chamber. Within the pouch or blister, the redox active molecule may be already in a developing solution or buffer, or upon opening or piercing a developing solution or buffer is released to solubilize or suspend the redox active molecule.

In an embodiment, the system of the present disclosure further comprises one or more washing portions positioned between the one or more hybridization portions and the redox portion. As used herein, the “washing portion” is intended to refer to a portion in which the magnetic bead complex is able to come into contact with a washing solution. The washing portion comprises or is capable of being loaded with the washing solution.

In an embodiment wherein the system includes a cartridge, the one or more washing portions may be one or more chambers located along the second passage or may be within the second passage itself.

In an embodiment wherein the system includes a strip, the one or more washing portions may be a region or regions on the strip downstream in a flow path from one or more hybridization portions and upstream of the redox portion. In such embodiments, by “interconnected” it is meant that the magnetic bead complex is able to flow from one portion or region of the strip to another.

In an embodiment, the washing solution may already be in the washing portion at the time the magnetic bead complex reaches this portion. In other embodiments, washing solution may be contained within a washing pouch or blister that is opened or pierced to washing solution into (e.g. for chambers or passages) or onto (e.g. for strips) the washing portion. In embodiments where the washing portion is a chamber or is performed within the second passage, the pouch or blister may be internal or external to the chamber or passage, so long as the washing solution is capable of being released into the chamber or passage.

As used herein, the term “washing solution” is intended to refer to any solution suitable for washing the magnetic bead complex to remove any undesired components residual either from the test sample itself or from the one or more hybridization chambers (e.g. unbound antibody). In an embodiment, the washing solution is an oil or organogel. Hydrophobicity and elastic properties of organogel may make it a good and suitable choice for efficient liquid-phase displacement. Also, organogel may assist in preventing the magnetic bead complex from flowing back into the previous portion. Those skilled in the art will appreciate that various alternative options for the washing solution are readily available.

The system of the present disclosure comprises a multitude of magnetic sources. As used herein, the “multitude of magnetic sources” is intended to refer to magnetic sources that are capable of providing a controlled magnetic field to direct the sequential movement of the magnetic beads and, as a consequence, the magnetic bead complex. The multitude of magnetic sources are positioned throughout the system (e.g. the device component) for moving the magnetic bead complex in a sequential movement from the one or more hybridization portions to the redox portions and optionally beyond into another portion, chamber or passage. The different magnetic sources of the multitude of magnetic sources are capable of being turned on and off in a desired sequence to achieve the sequential movement of the magnetic bead complex. In this regard, in an embodiment, the system or device comprises a processor and/or circuitry for controlling the timed sequential activation and de-activation of the different magnetic sources.

The magnetic sources may be any suitable component that provides or is capable of conducting a magnetic charge. In an embodiment, the magnetic sources are electromagnets, magnet wire, magnets, or any combination thereof. In an embodiment, the magnetic sources are placed in a zig zag pattern throughout the system or a device of the system.

Advantageously, the movement of the magnetic beads in a sequential movement and/or in different portions or routes in a 3-dimensional space (e.g. of the hybridization chamber), in particular for example in a zig zag pattern for increased travel distance, can create a swishing and/or mixing movement. During the sequential and/or spatial movements, including the swishing/mixing movement of the magnetic beads, improved hybridization may be achieved with a rapid and efficient mixing (e.g. with the hybridization solution), improved washing may be achieved, and an improved redox reaction may be achieved, all providing for increased efficiency and accuracy in detecting a target analyte in the test sample. This directed path of sequential and/or spatial movement may be designed and achieved by turning on and off the magnetic sources. In an embodiment, the magnetic beads move in bulk and travel together. In particular, travelling in bulk movement while mixing or swishing facilitates hybridization and washes. In another embodiment, the magnetic beads can travel by alternate routes or at different rates of speed, for example based on characteristics of the magnetic beads and/or bound material. For example, in some embodiments, the systems and methods herein may involve the use of magnetic beads made from different materials (e.g. having different magnetic properties allowing weaker or stronger interactions with the magnetic field) and/or magnetic beads of different sizes. In some embodiments, the magnetic beads may move at different rates based on other properties, such as based on the particular target analyte bound thereto (or lack thereof).

In an embodiment, the system further comprises one or more valves capable of being opened and closed. This feature is of particular relevance to systems and cartridge embodiments disclosed herein having passages and chambers. As used herein, the “valves” are intended to refer to any structure that is capable of being opened and closed to control fluid flow. The one or more valves may be located within the first passage, the second passage, or both. Advantageously, the valves, when closed, prevent materials or fluid to pass (e.g. prevent backflow into a previous chamber or passage).

In an embodiment, the system further comprises a Faraday cage separating the electrode set and/or the potentiostat from the multitude of magnetic sources to prevent interference. The Faraday cage is an enclosure used to block electromagnetic fields. Advantageously, this may assist in preventing any interference between the magnetic sources and the potentiostat. Those skilled in the art will appreciate that various alternative structures or materials are readily available to achieve the same goal.

The system of the present disclosure includes an electrode set. As used herein, the “electrode set” is intended to refer to a set of multiple electrodes for measuring the energy properties of the test sample received therein. In operation, and without being bound to theory, the electrode set is capable of being connected to and/or electrically engage with a potentiostat to perform amperometry to obtain an electrochemical measurement based on the reduction of an oxidized form of a substrate back to its original form (after the enzyme oxidizes the substrate in the redox portion / chamber), and a potential is applied to the working electrode where the current is then recorded.

In an embodiment, the electrode set comprises a three-electrode cell. In an embodiment, the three-electrode cell comprises a reference electrode, a counter electrode and a working electrode. In an embodiment, the working electrode and the counter electrode are comprised of a carbon ink, and the reference electrode is comprised of Ag, AgCl or a carbon ink. In a particular embodiment, the working electrode and the counter electrode are both comprised of the carbon ink, and the reference electrode is comprised of an Ag, AgCl ink. In particular, Ag, AgCl containing reference electrodes may provide for a more stable and reliable reference potential. In an embodiment, all three of the electrodes are made of the same material. In an embodiment, all three of the electrodes are a printed carbon ink. In an embodiment, the electrodes may contain or be coated with an acid or acid solution. In a particular embodiment, the working electrode may be coated with an acid, for example to stop or quench the redox reaction. By coating the working electrode with an acid (e.g. sulfuric acid), upon contact of the reactant solution from the redox portion with the working electrode, the acid is dissolved and the redox reaction is substantially stopped or quenched, and more particularly is fully stopped or quenched (before amperometry is performed). In particular, without being bound by theory, the acid denatures the enzyme and stops the enzymatic activity and conversion. Furthermore, the acid also converts the redox molecule to a different form. After the redox reaction associated with the enzyme is stopped or quenched, amperometry is the reverse redox reaction measuring how much has been converted, thereby allowing for quantitative measurement of the test sample target analyte concentration.

As used herein, by “reactant solution” it is meant to refer to the developing solution in the redox portion after reaction with the magnetic bead complex. In an embodiment, the mixture is allowed to react for a period of between 5 seconds and 5 minutes before being stopped. In an embodiment, the mixture is allowed to react for a period of about 5 seconds, 10 seconds, 15 seconds, 20 seconds, 25 seconds, 30 seconds, 35 seconds, 40 seconds, 45 seconds, 50 seconds, 55 seconds, 1 minute, 1.5 minutes, 2 minutes, 2.5 minutes, 5.0 minutes, 7.5 minutes, or 10 minutes. In an embodiment, the mixture is allowed to react for a period of about 30 seconds.

The system of the present disclosure comprises a potentiostat. As used herein, the “potentiostat” is intended to refer to a potentiostat connected, connectable or electrically engageable to the electrode set for performing amperometry to obtain an electrochemical measurement based on conversion of the redox active molecule by the enzyme on the second antibody. In an embodiment, the potentiostat comprises a potentiostat circuitry electrically engaging the electrode set for performing amperometry to obtain the electrochemical measurement.

The potentiostat is connected or electrically engaged to the electrode set for analyzing the presence, absence, amount and/or concentration of target analyte in the test sample. In general, the potentiostat is designed to control the working electrode's potential in the three-electrode cell. The potentiostat can contain many internal circuits that allow it to function in this capacity. The circuits generate and measure potentials and currents. An exemplary potentiostat that may be used as part of an electrochemical-sensor system is disclosed in International Patent Application Serial No. PCT/CA2019/051567 and United States Patent Publication No. US 2021/0270766 A1, in which the electrochemical-sensor system can be modified to fit the system disclosed in the present disclosure. In particular, similar to the electrochemical-sensor system disclosed therein, the potentiostat of the present disclosure may comprise a potentiotat circuitry electronically engaged with the electrode set for measuring the energy properties of the magnetic bead complex or the reactant solution. In particular, in one embodiment, the potentiostat circuitry is in the form of a Direct-Current (DC) potentiostat circuitry used to control the voltage between the working electrode (WE) and reference electrode (RE).

In an embodiment of the present disclosure, amperometry may be performed with the reactant solution remaining in the redox chamber. In such embodiments, a stop solution comprising an acid may be added to the redox chamber to stop the reaction. Further, in such embodiments, the working electrode of the three-electrode set may by in contact with or operationally exposed to the redox chamber. In an embodiment, the working electrode may be coated with an acid to stop the redox reaction.

In another embodiment, amperometry may be performed by moving the reactant solution from the redox chamber to another component of the system, cartridge or strip. In an embodiment, the system, cartridge or strip of the present also includes an electrode chamber, passage, or portion that comprises, or is operationally associated with, a working electrode of the electrode set. In such embodiments, a stop solution comprising an acid may be added to the redox chamber before moving the reactant solution onwards. In other embodiments, a stop solution comprising an acid may be located within or added to the electrode chamber, passage, or portion. In other embodiments, the working electrode may be coated with an acid to stop the redox reaction.

As described herein, the system of the present disclosure may be a fixed unit or may comprise a cartridge or strip and a device. It will be understood that any description herein relating to structures or embodiments of the cartridge (e.g. chambers, passages, etc.) may equally apply to a system herein that is a fixed unit. Likewise, the fixed unit may include a permanently embedded strip, and therefore any description herein relating to structures or embodiments of the strip may equally apply to a system herein that is a fixed unit.

In another aspect, the present disclosure relates to a disposable cartridge comprising: a port or receptacle for receiving a test sample; one or more hybridization chambers interconnected to the port or receptacle by a first passage, wherein the one or more hybridization chambers comprise within or are capable of being loaded with: a first antibody coupled to a magnetic bead, the first antibody capable of specifically binding to the target analyte, and a second antibody coupled to an enzyme, the second antibody capable of specifically binding to the target analyte and the enzyme is capable of converting a redox active molecule, wherein the first antibody and the second antibody are from an antibody source located on the cartridge; a redox chamber interconnected to the one or more hybridization chambers by a second passage, the redox chamber comprising within or capable of being loaded with a developing solution having the redox active molecule; and an electrode set; wherein the cartridge is configured for sequential movement of the magnetic bead from the hybridization chamber to the redox chamber and optionally beyond, by a multitude of magnetic sources present in a device capable of housing the cartridge.

By “disposable”, it is meant that the cartridge may be for single use application. The cartridge may be of any shape or configuration suitable for use in the device. In an embodiment, the cartridge has a generally linear path of flow. In an embodiment, the cartridge has a non-linear (e.g. circular) path of flow. Terms and components used in the above description of the cartridge have similar meaning as used elsewhere herein in describing the system of the present disclosure.

In another embodiment, the present disclosure relates to a disposable strip comprising: a test sample receiving portion; one or more hybridization portions downstream of the test sample receiving portion, wherein the one or more hybridization portions comprise or are capable of being loaded with: a first antibody coupled to a magnetic bead, the first antibody capable of specifically binding to the target analyte, and a second antibody coupled to an enzyme, the second antibody capable of specifically binding to the target analyte and the enzyme is capable of converting a redox active molecule; wherein the first antibody and the second antibody are from an antibody source located on the strip; a redox portion downstream of the hybridization portion, the redox portion comprising or capable of being loaded with a developing solution having the redox active molecule; and an electrode set; wherein the strip is configured for sequential movement of the magnetic bead from the hybridization chamber to the redox chamber and optionally beyond, by a multitude of magnetic sources present in a device capable of housing the strip.

By “disposable”, it is meant that the strip may be for single use application. The strip may be of any width, length or design suitable for use in the device. In an embodiment, the strip has a generally straight-line linear path of flow. Terms and components used in the above description of the strip have similar meaning as used elsewhere herein in describing the system of the present disclosure.

With reference below to the drawings herein, non-limiting embodiments of system, cartridge and strips of the present disclosure are described.

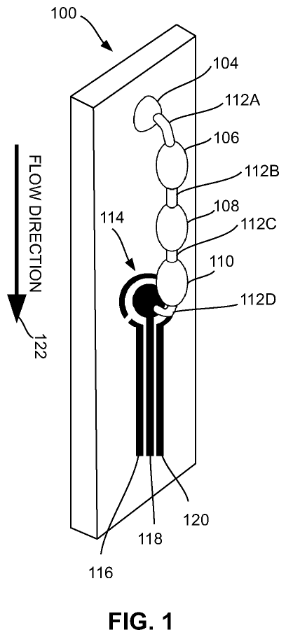

Turning now to FIG. 1, a cartridge is generally identified using reference numeral 100. The cartridge 100 in this embodiment, is a linear design, and from upstream to downstream according to the flow direction (indicated by the arrow 122), comprises a test sample receiving portion 104 for receiving the test sample, a hybridization portion 106, a washing portion 108, a redox portion 110, and an electrode set 114 with a three-electrode cell.

Various portions of the cartridge 100, such as the test sample receiving portion 104, the hybridization portion 106, the washing portion 108, the redox portion 110, and the electrode set 114, are interconnected by passages 112A, 112B, 112C and 112D. The passages can be tubing or microchannels. Furthermore, in this particular embodiment, the portions 104, 106, 108, and 110 are chambers.

In an embodiment, the cartridge 100 further comprises one or more valves located within the passages (not shown) capable of being opened and closed. The valves, when closed, can prevent materials or fluid from passing (e.g. prevent backflow into a previous chamber or passage). The valves can also be opened, when needed, to direct or allow the flow to proceed through the cartridge 100.

The test sample receiving portion 104 can optionally comprise a size-selective mesh (not shown) that may be used to trap undesired molecules or components or the test sample from passing through the cartridge 100. In an embodiment, the test sample receiving portion 104 comprises a size selective mesh for prohibiting cells or molecules larger than a defined size to pass through. In an embodiment, the size selective mesh has a defined size of 4.5 μm. Furthermore, different mesh materials may be used to try to trap or capture different undesired materials. For example, the size selective mesh may have a defined size that absorbs and does not allow large complexes or biomolecules (e.g. of a megadalton size) to pass through, but does allow the target analyte to pass. The test sample receiving portion 104 may also be made of any suitable material to receive the test sample and permit fluid flow, e.g. by capillary action. Those skilled in the art will appreciate that various alternative materials or mesh sizes are readily available.

The hybridization portion 106 is a portion of the cartridge 100 in which the test sample is able to come into contact with a first antibody coupled to a magnetic bead and capable of specifically binding to the target analyte, and a second antibody coupled to an enzyme and also capable of specifically binding to the target analyte. The enzyme is one that is capable of converting a redox active molecule. In particular, the enzyme is capable of converting a redox molecule to a form that changes its “energy properties” and that redox molecule later is detected with the potentiostat. In an embodiment, the hybridization portion 106 is a chamber.

The hybridization portion 106 is interconnected to the test sample receiving portion 104 by a first passage 112A to allow the test sample to move to the hybridization chamber 106. The first passage 112A allows materials to pass through and link the various portions and chambers of the device together. In an embodiment, the passage is a tubing or a microchannel.

In an embodiment, the first antibody and the second antibody are in a hybridization solution, and the hybridization solution is contained in a hybridization pouch or blister capable of being pierced to release the hybridization solution into the hybridization portion 106. The pouch or blister is any structure capable of being opened or pierced to release its contents. Those skilled in the art will appreciate that various materials for the pouch or blister are readily available. In an embodiment, the system of the present disclosure may comprise piercing components for piercing the pouch or the blister to release the hybridization solution.

In an embodiment, the first passage 112A or the hybridization portion 106 further comprise an absorbent pad (not shown) for absorbing a fluid and transferring it into the hybridization chamber 106. While the magnetic beads coupled to the first antibody can continue to move in a sequential movement and travel via a directed path achieved by turning on and off the magnetic sources, non-complexed materials will be left behind and/or trapped to the absorbent pad. This provides an efficient wash, even without a washing portion and a washing solution.

The washing portion 108, is a portion of the cartridge 100 in which the magnetic bead complex is able to come into contact with a washing solution. In an embodiment, the washing portion 108 is positioned along the second passage (112B/112C) between the hybridization portion 106 and the redox portion 110. Therefore, the washing function can be provided by the washing chamber 108 and/or within the second passage (112B/112C).

The washing solution can be any solution suitable for washing the magnetic bead complex as described herein. In an embodiment, the washing solution is an oil or organogel.

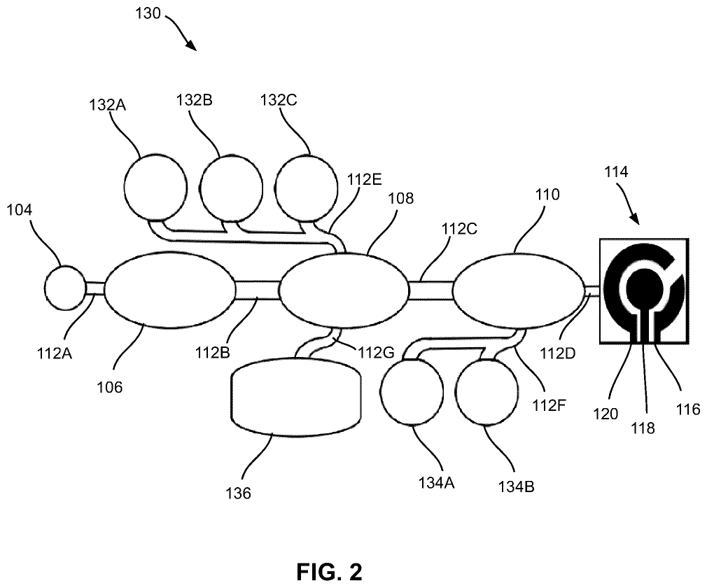

In an embodiment, the washing solution is contained in a washing pouch or blister capable of being pierced to release the washing solution (see FIG. 2). The pouch or blister is any structure capable of being opened or pierced to release its content. Those skilled in the art will appreciate that various materials for the pouch or blister are readily available. In an embodiment, the system further comprises piercing components for piercing the pouch or the blister to release the washing solution.

The redox portion 110, is a portion of the device in which the magnetic bead complex is contacted with a developing solution having redox active molecules. The redox portion 110 is interconnected to the hybridization chamber 106 by a second passage (112B/112C). The redox portion 110 comprises or is capable of being loaded with a developing solution having the redox active molecules. In an embodiment, the developing solution is contained in a developing pouch or blister capable of being pierced to release the developing solution into the redox portion (see FIG. 2). In an embodiment, the redox portion 110 is a chamber. The pouch or blister is any structure capable of being opened or pierced to release its content. Those skilled in the art will appreciate that various materials for the pouch or blister are readily available. In an embodiment, the system comprises piercing components for piercing the pouch or the blister to release the developing solution.

The electrode set 114 is a set of multiple electrodes downstream of or extending into the redox portion for measuring the energy properties of the magnetic bead complex or reactant solution and is connected to a potentiostat to perform amperometry to obtain an electrochemical measurement based on the conversion of the redox active molecules. In this embodiment, the electrode set 114 comprises a three-electrode cell with a working electrode 118, a reference electrode 116 and a counter electrode 120. The reference electrode 116 may comprise Ag, AgCl, or a carbon ink. The working electrode 118 and the counter electrode 120 comprises a carbon ink. The electrodes, and in particular the working electrode 118, may be coated with an acid (e.g. sulfuric acid) to stop the redox reaction. In particular, with the working electrode 118 coated with an acid, as soon as the reactant solution reaches the electrode, the acid is dissolved and the redox reaction is stopped (before amperometry is performed).

Turning now to FIG. 2, a cartridge is generally identified using reference numeral 130. The cartridge in this embodiment, is generally similar to the cartridge 100. The cartridge 130 comprises multiple pouches or blisters capable of being pierced to release the content inside the pouches or blisters.

The washing portion/chamber 108 is connected to three pouches or blisters 132A, 132B and 132C via passage 112E, which is similar to passages 112A to 112D shown in FIG. 1. The pouches or blisters 132A, 132B and 132C contain washing solutions or washing buffers. The pouches or blisters 132A, 132B and 132C may contain the same or different washing buffers. The washing portion/chamber 108 is also connected to a waste portion/chamber 136, for containing any waste resulted from the wash. Once the pouches or blisters 132A, 132B and 132C are pierced, their contents will be released into the washing portion 108.

The redox portion/chamber 110 is connected to two pouches or blisters 134A and 134B via passage 112F, which is similar to passages 112A to 112D shown in FIG. 1. One of the pouches or blisters 134A and 134B contains a developing solution or developing buffer with the redox active molecule the substrate and the other pouch or blister contains a stopper solution (e.g. a solution containing acid such as sulfuric acid) to stop or quench the redox reaction. Once the pouches or blisters 134A and 134B are pierced, their contents will be released into the redox portion 110.

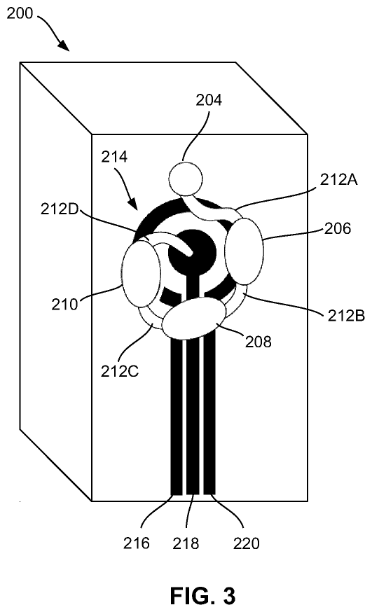

Turning now to FIG. 3, a cartridge is generally identified using reference numeral 200. The cartridge 200 in this embodiment, is generally similar to the cartridge 100, but is a circular design. The cartridge 200 comprises a port or receptacle 204 for receiving the test sample, a hybridization chamber 206, a washing chamber 208, a redox chamber 210, and an electrode set 214 with a three-electrode cell.

The electrode set 214 comprises a three-electrode cell with a working electrode 218, a reference electrode 216 and a counter electrode 220. As above, the reference electrode 116 may comprise Ag, AgCl, or a carbon ink. The working electrode 118 and the counter electrode 120 comprise a carbon ink. The electrodes, and in particular the working electrode 218, may be coated with an acid. In particular, with the working electrode 218 coated with an acid, as soon as the test sample solution reaches the working electrode 218, the acid is dissolved and the redox reaction is stopped (before amperometry is performed).

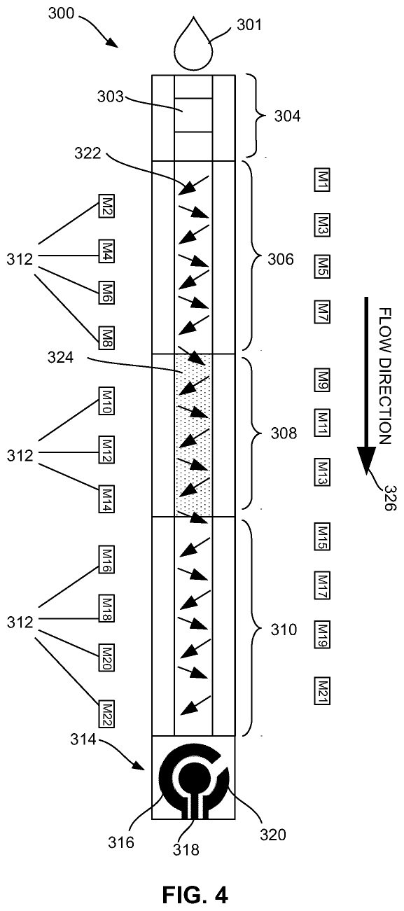

Turning now to FIG. 4, a strip is generally identified using reference numeral 300. The strip 300 in this embodiment, is generally similar to the cartridge 100, but is a strip design. The strip 300, from upstream to downstream according to the flow direction (indicated by the arrow 326), comprises a test sample receiving portion 304 for receiving the test sample 301, a hybridization portion 306, a washing portion 308, a redox portion 310, and an electrode set 314 with a three-electrode cell. The test sample receiving portion 304 comprises a size-selective mesh 303 as described herein that will stop and trap any undesired particles. The system as disclosed herein, and in particular embodiments a device into which the strip 300 may be inserted, comprises a multitude of magnetic sources 312 located throughout the system or device (not located on the strip 300). In an embodiment, the strip 300 comprises an absorbent pad as the test sample receiving portion 304.

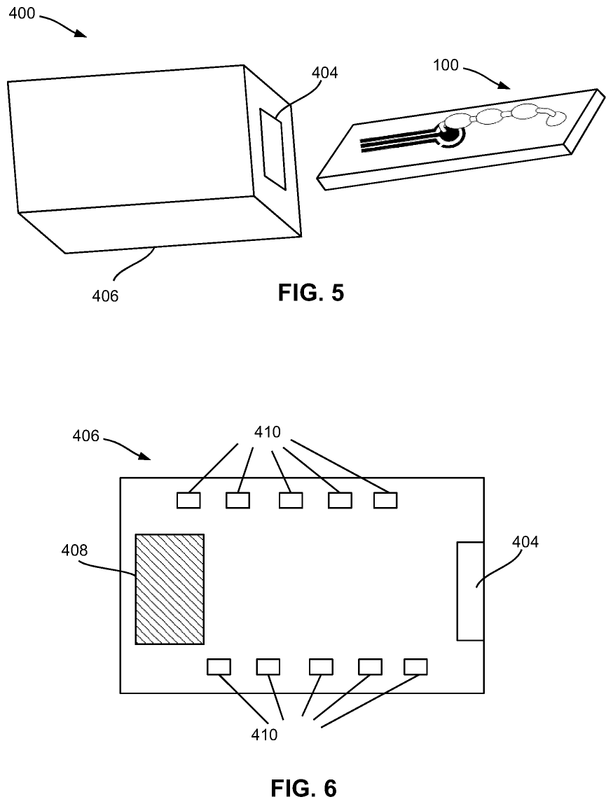

The multitude of magnetic sources 312 are magnetic sources that are capable of providing a controlled magnetic field to direct the sequential movement of the magnetic beads. The multitude of magnetic sources 312 are positioned throughout the system or device (see e.g. FIGS. 5 and 6, device 406) for moving the magnetic bead complex in a sequential movement from the hybridization portion 106/206/306 to the redox portion 110/210/310 and optionally beyond, wherein different magnetic sources of the multitude of magnetic sources 312 are capable of being turned on and off in a desired sequence to achieve the sequential movement of the magnetic bead complex. In an embodiment, the magnetic sources are electromagnets.

In this embodiment, the magnetic sources 312 are placed in a zig zag pattern throughout the system or device. The zig zag pattern of sequential movement of the magnetic bead complex is shown by the arrows 322. Advantageously, a zig zag pattern allows the magnetic bead complex to move in a zig zag sequential movement throughout the device and provides a greater swishing and/or mixing movement and a further travel distance resulting in improved hybridization, washing, and/or redox reaction.

The electrode set 314 comprises a three-electrode cell with a working electrode 318, a reference electrode 316 and a counter electrode 320. As above, the reference electrode 116 may comprise Ag, AgCl, or a carbon ink. The working electrode 118 and the counter electrode 120 comprise a carbon ink. The electrodes, and in particular the working electrode 318, may be coated with an acid to stop the redox reaction. In particular, with the working electrode 318 coated with an acid, as soon as the reactant solution reaches the electrode, the acid is dissolved and the redox reaction is stopped (before amperometry is performed).

Turning now to FIG. 5, a system 400 is shown, comprising the cartridge 100 and a device 406 capable of housing the cartridge 100. In an embodiment, the cartridge can be cartridge 200 (circular design) or strip 300. Furthermore, the cartridge or strip is removable from the device 406 or disposable.

Turning now to FIG. 6, a cross-sectional view of the device 406 is shown. The device 406 contains a multitude of magnetic sources 410 (see FIG. 6), a potentiostat 408 and a slot or opening 404 for receiving the cartridge 100. The magnetic sources are the same or similar to the magnetic sources described herein and shown in FIG. 4.

According to another aspect of the present disclosure, there is provided a kit for detecting a target analyte in a test sample, the kit comprising: the device as disclosed herein and instructions for using the device with a cartridge or strip as disclosed herein. In an embodiment, the kit further comprises one or more cartridges or strips as disclosed herein. In an embodiment, the kit further comprises one or more buffers or solutions, including for example solutions for solubilizing or suspending the test sample, solutions comprising the first and/or second antibody, wash buffers or solutions, redox buffers or solutions, solutions containing an acid, or any combination thereof.

### Methods

In an embodiment, the present disclosure relates to a method for detecting a target analyte in a test sample, the method comprising: providing the system as described herein; contacting a test sample with the test sample receiving portion for receiving the test sample; allowing the test sample to migrate from the test sample receiving portion to the one or more hybridization portions; contacting the test sample, within the one or more hybridization portions, with the first antibody and the second antibody; sequentially activating the different magnetic sources of the multitude of magnetic sources to move the magnetic bead coupled to the first antibody from the one or more hybridization portions to the redox portion; contacting the magnetic beads in the redox portion with a developing solution having the redox active molecule; performing amperometry using the potentiostat to obtain an electrochemical measurement from the electrode set based on conversion of the redox active molecule by a redox reaction; and determining or evaluating the presence, absence and/or quantity of the target analyte based on the electrochemical measurement.

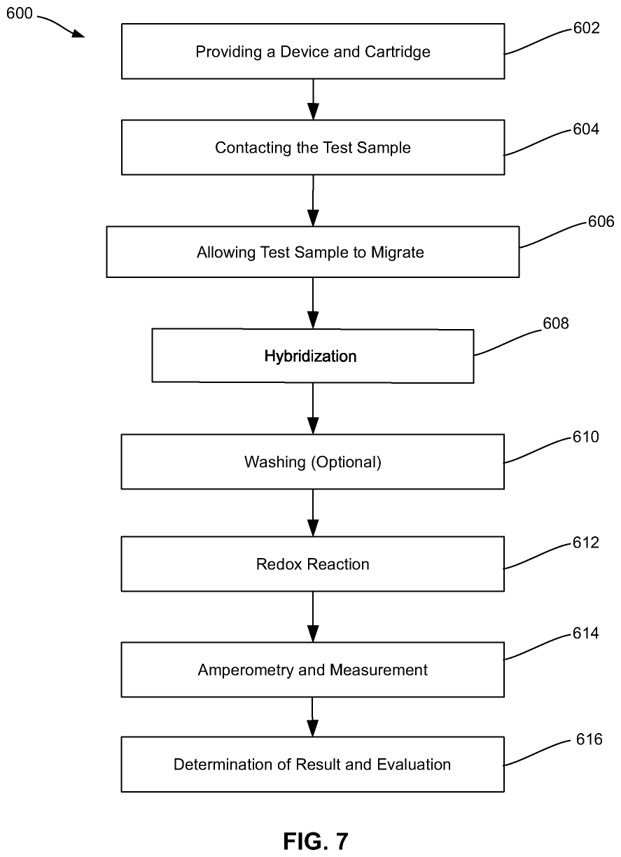

FIG. 7 is a flowchart showing the steps of a method 600 for detecting a target analyte in a test sample, according to one embodiment of this disclosure. The method 600 begins when a cartridge and a device is provided (step 602). At step 604, a test sample is contacted with the cartridge, in particular, the cartridge's test sample receiving portion. For example, dropping one or more drops of the fluid test sample onto the test sample receiving portion 104 of the cartridge 100 or the test sample receiving portion 304 of the strip 300. The cartridge can be a circular design, a linear design or a strip (see e.g. FIGS. 1 to 4).

At step 606, the test sample is allowed to migrate (e.g. by capillary movement from the test receiving portion to the hybridization portion) through the cartridge or the strip towards the hybridization portion 106 or 306. In an embodiment, at step 606, when the test sample is migrating from the test sample receiving portion to the one or more hybridization portions, the test sample also passes through a size selective mesh at the test sample receiving portion. For example, mesh 303 in FIG. 4.

At step 608, the hybridization step or phase, if the target analyte is present in the test sample, the magnetic beads coupled to a first antibody will specifically bind to the target analyte. Furthermore, the second antibody coupled to the enzyme that is capable of converting a redox active molecule will also specifically bind to the target analyte. In preferred embodiments, both the first antibody and the second antibody are capable of specifically binding to the target analyte at different regions of the target analyte.

In an embodiment, the first antibody and/or the second antibody are in a hybridization solution, and the hybridization solution is contained in a hybridization pouch or blister capable of being pierced to release the hybridization solution into the one or more hybridization portions. Thus, at step 608, there may be an additional step of piercing one or more hybridization pouches or blisters to release the first antibody and/or second antibody into the hybridization portion. This piercing step may be achieved by a piercing component within the device.

In another embodiment, the first antibody and second antibody are lyophilized and they are contained in the hybridization portion. In such embodiment, a supportive buffer mix is added to the hybridization portion to solubilize the antibodies.

During and after step 608, the magnetic bead complex is moving in a directed path from the sequential movement achieved by turning on and off the magnetic sources.

At step 610, the optional washing step or phase, the magnetic bead complex, is being washed with a washing solution. In an embodiment, the washing step 610 is performed within washing portion 108 or 306.

In another embodiment, the washing step 610 is performed within the second passage (112B and/or 112C) connecting the hybridization portion and the redox portion and a washing chamber is not required as the magnetic bead complex is washed as it moves through the second passage (112B and/or 112C). Movement is again in a directed path from the sequential activation achieved by turning on and off the magnetic sources. In an embodiment, the washing solution is oil or organogel.

In an embodiment, the washing solution is contained in a washing pouch or blister capable of being opened or pierced to release the washing solution (see FIG. 2 for example). Thus, at step 610, there may be an additional step of piercing the washing pouch or blister to release the washing solution into the second passage or the washing portion. This piercing step may be achieved by a piercing component within the device.

Washing step 610 is optional, because during and also after step 608, the magnetic beads are travelling in a directed path (e.g. zig zag pattern) with a swishing and/or mixing movement likewise achieve a washing, in effect. Again, only the magnetic bead complex will be moved through the device by the sequence movement achieved by turning on and off the magnetic sources. Thus, in certain embodiments, even without a washing solution, efficient washing can be achieved by allowing the magnetic beads to travel through the cartridge or the strip.

At step 612, the redox step or phase, the magnetic bead complex travels toward a redox portion 110 or 310, which contains or is capable of being loaded with a developing solution having redox active molecules. The enzyme, coupled to the second antibody (binding with the target analyte), will convert the redox active molecule to a form that changes its “energy properties”, to be later detected.

In an embodiment, the developing solution is contained in a developing pouch or blister capable of being opened or pierced to release the developing solution into the redox portion (see FIG. 2 for example). Thus, at step 612, there may be an additional step of piercing the developing pouch or blister to release the developing solution into the redox portion. This piercing step may be achieved by a piercing component on the device.

At step 616, the amperometry and measurement step or phase, the reactant solution is exposed to an electrode set 114 or 314, which is connected or electronically engaged to a potentiostat 408. In an embodiment, the electrode set 114 or 314 is a three-electrode cell with a reference electrode, a counter electrode and a working electrode.

Before or during step 616, the redox reaction at step 612 needs to be stopped. For stopping the redox reaction, in an embodiment, the working electrode is exposed to the redox portion or chamber, where a stopper solution (e.g. a solution containing acid such as sulfuric acid) is added to stop the redox reaction before amperometry is performed.

In another embodiment, the working electrode is exposed to a channel or passage that exits the redox portion or chamber, where the contents of the redox portion can be released into the passage onto the working electrode and the working electrode is coated with a stopper solution material (e.g. acid) to stop the redox reaction, and then amperometry can be performed.

In another embodiment, the stopper solution is first added to the redox portion or chamber and the redox reaction is stopped. Then, the content is released from the redox portion or chamber into a passage or channel connecting to the electrode set.

After the redox reaction is stopped and the electrode set contacts the reactant solution from the redox portion, amperometry is performed. In an embodiment, amperometry is performed for about 30 seconds. The electrode set is connected or electronically engaged to a potentiostat for performing amperometry to obtain an electrochemical measurement based on conversion of the redox active molecule. In an embodiment, the potentiostat further comprises a potentiostat circuitry electrically engaging the electrode set for performing amperometry to obtain the electrochemical measurement.

In an embodiment, the step 616 of performing amperometry also comprises flowing the contents of the redox portion to an electrode chamber, passage or portion that comprises, or is operationally associated with, a working electrode of the electrode set.

After performing amperometry, measurements are obtained and results are determined based on the measurements and an evaluation may be provided with respect to the presence or absence of the target analyte in the test sample, the concentration of the target analyte, or both.

From step 608 to step 614, the different magnetic sources of the multitude of magnetic sources are sequentially activated to move the magnetic bead complex from the hybridization portion to the redox portion. In an embodiment, the multitude of magnetic sources comprises sequentially activating the different magnetic sources in a zig zag pattern along a path of the sequential movement. As described herein, this increases the travel distance and provide a swishing and/or mixing movement that improves the hybridization, washing and redox phase.

In another embodiment, from step 606 to step 614, one or more valves (located within the passages 112A to 112D) are closed to prevent fluid backflow to the previous portion, chamber or passage. The valves are any valve that is capable of being opened and closed and are used to control fluid flow forward and/or backwards.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

It should be understood that the compositions and methods are described in terms of “comprising,” “containing,” or “including” various components or steps, the compositions and methods can also “consist essentially of or ”consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. A system for detecting a target analyte in a test sample, the system comprising:
a test sample receiving portion;
one or more hybridization portions interconnected to the test sample receiving portion, wherein the one or more hybridization portions comprise or are capable of being loaded with:
a first antibody coupled to a magnetic bead, the first antibody capable of specifically binding to the target analyte, and
a second antibody coupled to an enzyme, the second antibody capable of specifically binding to the target analyte, wherein the enzyme is capable of converting a redox active molecule;
a redox portion interconnected to the one or more hybridization chambers, wherein the redox portion comprises or is capable of being loaded with a developing solution having the redox active molecule;
an electrode set;
a potentiostat for performing amperometry to obtain an electrochemical measurement based on conversion of the redox active molecule; and
a multitude of magnetic sources positioned for moving the magnetic bead in a sequential movement from the hybridization portion to the redox portion and optionally beyond, wherein different magnetic sources of the multitude of magnetic sources are capable of being turned on and off in a desired sequence to achieve the sequential movement of the magnetic bead.

2. The system of claim 1, comprising:
a cartridge or a strip that is insertable and removable from a device, the cartridge or strip comprising the test sample receiving portion, the one or more hybridization portions, the redox portion, and the electrode set; and
the device comprising the potentiostat and the multitude of magnetic sources.

3. (canceled)

4. The system of claim 2, comprising the cartridge, wherein the cartridge comprises:
a port or receptacle as the test sample receiving portion;
one or more hybridization chambers as the one or more hybridization portions, interconnected to the port or receptacle by a first passage;
a redox chamber as the redox portion interconnected to the one or more hybridization chambers by a second passage; and
the electrode set.

5. The system of claim 4, wherein the cartridge further comprises one or more valves capable of being opened and closed, the one or more valves located within the first passage, the second passage, or both.

6. The system of claim 4, wherein the first passage, the second passage, or both, is a tubing or a microchannel.

7. The system of claim 2, comprising the strip, wherein the strip comprises an absorbent pad as the test sample receiving portion.

8. (canceled)

9. The system of claim 1, wherein the first antibody and/or the second antibody are in a hybridization solution, and the hybridization solution is contained in a hybridization pouch or blister capable of being pierced to release the hybridization solution into or onto the one or more hybridization portions.

10. The system of claim 1, wherein the one or more hybridization portions comprise:
a first hybridization portion interconnected to the test sample receiving portion and comprising or capable of being loaded with the first antibody; and
a second hybridization portion interconnected downstream to the first hybridization portion and comprising or capable of being loaded with the second antibody.

11. The system of claim 1, which further comprises a washing portion positioned between the one or more hybridization portions and the redox portion, the washing portion comprising or capable of being loaded with a washing solution.

12. The system of claim 4, which further comprises a washing portion positioned along the second passage between the one or more hybridization portions and the redox portion, the washing portion comprising or capable of being loaded with a washing solution.

13. The system of claim 12, wherein the washing portion is a washing chamber.

14. The system of claim 11, wherein the washing solution is contained in a washing pouch or blister capable of being pierced to release the washing solution into or onto the washing portion.

15. (canceled)

16. The system of claim 1, wherein the developing solution is contained in a developing pouch or blister capable of being pierced to release the developing solution into or onto the redox portion.

17. (canceled)

18. The system of claim 2, wherein the device comprises one or more piercing components.

19. The system of claim 1, wherein the test sample receiving portion comprises a size selective mesh for capturing cells or molecules larger than a defined size.

20. (canceled)

21. The system of claim 1, wherein the test sample receiving portion or the one or more hybridization portions comprise an absorbent pad for absorbing a fluid and transferring it into the one or more hybridization portions.

22. The system of claim 1, wherein the electrode set comprises a three-electrode cell, and the three-electrode cell comprises a reference electrode, a counter electrode and a working electrode.

23. (canceled)

24. The system of claim 22, wherein the working electrode and the counter electrode are comprised of a carbon ink, the reference electrode is comprised of Ag, AgCl or the carbon ink, and optionally the working electrode is further coated with an acid.

25. The system of claim 1, wherein the magnetic sources are electromagnets.

26. The system of claim 1, wherein the magnetic sources are positioned in a zig zag pattern along a path of the sequential movement.

27.-45. (canceled)
